(19) United States
(12) Patent Application Publication
(10) Pub. No.: US 2023/0118955 A1
(43) Pub. Date: Apr. 20, 2023

(54) VIRAL TREATMENTS INVOLVING MANUKA HONEY AND COMPONENTS THEREOF

(71) Applicant: MANUKAMED LIMITED PARTNERSHIP, MASTERTON (NZ)

(72) Inventors: CHRISTOPHER DAVID MCMAHON, HAMILTON (NZ); DENIS WATSON, MASTERTON (NZ)

(73) Assignee: MANUKAMED LIMITED PARTNERSHIP, MASTERTON (NZ)

(21) Appl. No.: 17/914,099

(22) PCT Filed: Apr. 1, 2021
(86) PCT No.: PCT/NZ2021/050057

§ 371 Date: Sep. 23, 2022

(30) Foreign Application Priority Data
Apr. 6, 2020 (NZ) 763277
Apr. 17, 2020 (NZ) 763668

(51) Int. Cl. A61K 35/644; A61P 31/14; A61K 31/121; A62B 23/02

(52) U.S. Cl. CPC A61K 35/644; A61P 31/14; A61K 31/121; A62B 23/025

(57) ABSTRACT

The use of Manuka Honey in the production of a medicament for treating a virus by applying the honey, via the medicament, to the periphery of tissue on which the virus is present. Further, The use of one or more of methylglyoxal; 3-phenyllactic acid; p-coumaric acid; quercetin; pinocembrin; pinobanksin; hesperidin; galangin; kaempferol; chrysin; and luteolin in the preparation of an antiviral medicament for administering to a patient to treat, or prevent, infection by the virus. Further, a respiratory mask comprising a barrier permeable to air, the barrier having sufficient Manuka Honey for substantially disinfecting a vims in air when breathed in or otherwise passed through the barrier while the mask is worn.

This application is a national stage of International Application No. PCT/NZ2021/050057, filed on Apr. 1, 2021, which claims priority to New Zealand Patent Application No. 763277 filed on Apr. 6, 2020, and New Zealand Patent Application No. 763668, filed on Apr. 17, 2020, all of which are hereby incorporated by reference in their entireties.

### FIELD OF THE INVENTION

This invention relates to treatments for viral infections, for example including but not limited to infections caused by coronaviridae. A particularly preferred form of the invention relates to a treatment for COVID-19 disease.

### BACKGROUND

Influenza and the non-influenza RNA respiratory viruses, which include respiratory syncytial virus, parainfluenza viruses, coronavirus, rhinovirus, and human metapneumovirus, are estimated to account for a quarter of viral deaths annually and, therefore, represent a considerable global burden to health systems.

The coronaviridae are the largest group of viruses belonging to the Nidovirales order. A key characteristic of these is that they are enveloped, non-segmented and have positive-sense RNA. The coronaviridae give rise to the common cold and to the Severe Acute Respiratory Syndrome (SARS) as well as Middle East Respiratory Syndrome viruses (MERS). In particular the coronavirus (SARS-CoV-2) that is responsible for COVID-19 disease has claimed many lives in a relatively short time span.

Viruses can be difficult to treat. Firstly, unlike most bacterial pathogens that exist outside the cells of a living being, viruses are intracellular parasites. They tend to be DNA or RNA that may or may not be contained in a protein envelope. Viruses are unable to replicate without a host cell. Consequently it is difficult to find compounds that can selectively target and block or prevent viral replication without interference to normal cellular processes, or without being toxic to the host. Secondly, each type of virus can only infect and parasitise a limited range of host cells, due to the recognition required between receptor cells on the host cell and the virus. For example, the human common cold generally only infects the cells lining the upper respiratory tract. Therefore, treatments that target epithelium in the upper respiratory tract alone are difficult to manufacture. Thirdly, some viruses are highly adaptable and readily mutate, which can negate the use of a specific drug or vaccines that were created to target the original virus or viral sequence.

### OBJECT OF THE INVENTION

It is an object of the invention per se to provide means for treating at least some viral infections. It is an object of a preferred embodiment of the invention to provide means for treating COVID-19 disease.

### Definitions

The term “comprising” if and when used in this document in relation to a combination of features should not be seen as excluding the option of additional unspecified features or steps. In other words the term should not be interpreted in a limiting way.

For the purposes of this document “Manuka Honey” means honey having at least 200 mg/kg 3-phenylactic acid (3-PLA), at least 1 mg/kg methoxyacetophenone, at least 1 mg/kg 2-methoxybenzoic acid, at least 1 mg/kg 4-hydroxyphenyllactic acid and at least 100 mg/kg methylglyoxal. Honey may be regarded as “Manuka Honey” even if not derived from a Manuka plant (Leptospermum scoparium), provided it meets the criteria noted above. For example, lower grade honey derived from the Manuka plant, and also non-Manuka plant honey, may each be appropriately spiked to become Manuka Honey.

### SUMMARY OF THE INVENTION

### Antiviral Medicaments

In one aspect, the invention comprises the use of Manuka Honey in the production of a medicament for treating a virus (eg in a human) by applying the honey, via the medicament, to the periphery of tissue on which the virus is present.

Optionally the virus comprises SARS-CoV-1, SARS-CoV-2 or MERS.

Optionally the virus causes COVID-19 disease.

Optionally the medicament has the honey in diluted form and is for topical application onto the tissue.

Optionally the medicament has the Manuka Honey in diluted form and is for application onto the back of the throat, nasal passages or mouth.

Optionally the medicament comprises the Manuka Honey in solution.

Optionally the medicament is for delivery to the tissue as a spray.

Optionally the medicament is for delivery to the tissue as an aerosol.

Optionally the medicament is for delivery via a nebuliser.

Optionally the Manuka Honey comprises methylglyoxal (MGO) at approximately 100-1,000 mg/kg of the honey.

Optionally the Manuka Honey comprises methylglyoxal (MGO) at approximately 200-1,000 mg/kg of the honey.

Optionally the Manuka Honey comprises methylglyoxal (MGO) at approximately >400 mg/kg of the honey.

Optionally the Manuka Honey comprises methylglyoxal (MGO) at ≥approximately 550 mg/kg of the honey.

Optionally the Manuka Honey comprises methylglyoxal (MGO) at ≥approximately 700 mg/kg of the honey.

Method of Treating Viral Diseases with Manuka Honey

In another aspect, the invention comprises a method for treating a viral disease (eg in a human), comprising applying Manuka Honey, directly or via a medicament, to the periphery of tissue on which the virus is present.

Optionally the virus comprises SARS-CoV-1, SARS-CoV-2 or MERS.

Optionally the virus causes COVID-19 disease.

Optionally the medicament has the Manuka Honey in diluted form and is applied onto the tissue topically.

Optionally the medicament has the Manuka Honey in diluted form and is applied onto the back of the throat, nasal passages or mouth.

Optionally the medicament comprises the Manuka honey in solution.

Optionally the medicament is delivered to the tissue as a spray.

Optionally the medicament is delivered to the tissue as an aerosol.

Optionally the medicament is delivered via a nebuliser.

Optionally the Manuka Honey comprises methylglyoxal (MGO) at approximately 100-1,000 mg/kg of the honey.

Optionally the Manuka Honey comprises methylglyoxal (MGO) at approximately 200-1,000 mg/kg of the honey.

Optionally the Manuka Honey comprises methylglyoxal (MGO) at approximately ≥400 mg/kg of the honey.

Optionally the Manuka Honey comprises methylglyoxal (MGO) at ≥approximately 550 mg/kg of the honey.

Optionally the Manuka Honey comprises methylglyoxal (MGO) at ≥approximately 700 mg/kg of the honey.

### Respiratory Mask

In a further aspect, the invention is a respiratory mask (eg for a human) comprising a barrier permeable to air, the barrier having sufficient Manuka Honey for substantially disinfecting a virus in air when breathed in or otherwise passed through the barrier while the mask is worn. The virus may or may not be airborne within droplets.

Optionally the mask is in the form of a respirator.

Optionally the Manuka Honey is effective in disinfecting SARS-CoV-1, SARS-CoV-2 or MERS viruses.

Optionally the Manuka Honey is effective in disinfecting viruses that cause COVID-19 disease.

Optionally the Manuka Honey comprises methylglyoxal (MGO) at approximately 100-1,000 mg/kg of the honey.

Optionally the Manuka Honey comprises methylglyoxal (MGO) at approximately 200-1,000 mg/kg of the honey.

Optionally the Manuka Honey comprises methylglyoxal (MGO) at approximately ≥400 mg/kg of the honey.

Optionally the Manuka Honey comprises methylglyoxal (MGO) at ≥approximately 550 mg/kg of the honey.

Optionally the Manuka Honey comprises methylglyoxal (MGO) at ≥approximately 700 mg/kg of the honey.

Optionally the barrier comprises fibrous sheet or pad that holds a layer of Manuka honey.

### Antiviral Medicament & Method Based on MGO,

In a further aspect the invention comprises the use of one or more of:

methylglyoxal;

3-phenyllactic acid;

p-coumaric acid;

quercetin;

pinocembrin;

pinobanksin;

hesperidin;

galangin; kaempferol;

chrysin; and

luteolin;

in the preparation of an antiviral medicament for administering to a patient to treat, or prevent, infection by the virus (eg in a human).

Optionally the virus comprises SARS-CoV-1, SARS-CoV-2 or MERS.

Optionally the virus causes COVID-19 disease.

Optionally the medicament comprises honey in diluted form and is for topical application onto tissue (eg human tissue).

Optionally the honey comprises Manuka Honey in diluted form and is for application onto the back of the throat, nasal passages or mouth.

Optionally the medicament comprises the Manuka Honey in solution.

Optionally the medicament is for delivery to the tissue as a spray.

Optionally the medicament is for delivery to the tissue as an aerosol.

Optionally the Manuka Honey comprises methylglyoxal (MGO) at approximately 100-1,000 mg/kg of the honey.

Optionally the Manuka Honey comprises methylglyoxal (MGO) at approximately 200-1,000 mg/kg of the honey.

Optionally the Manuka Honey comprises methylglyoxal (MGO) at approximately ≥400 mg/kg of the honey.

Optionally the Manuka Honey comprises methylglyoxal (MGO) at ≥approximately 550 mg/kg of the honey.

Optionally the Manuka Honey comprises methylglyoxal (MGO) at ≥approximately 700 mg/kg of the honey.

### DRAWINGS

Some preferred embodiments of the invention will now be described by way of example and with reference to the accompanying drawings, of which:

FIG. 1 graphically illustrates the antiviral effect of Manuka Honey on the virus H1N1;

FIG. 2 shows the immunofluorescent signal identified in samples of infected cells infected the with virus (H1N1);

FIGS. 3-7 Illustrate various test results obtained from tests run using methylglyoxal in relation to virus'; and

FIG. 8 illustrates a medical face mask having a Manuka Honey impregnated antiviral barrier layer, prior to assembly.

### DETAILED DESCRIPTION

### H1N1 Trials

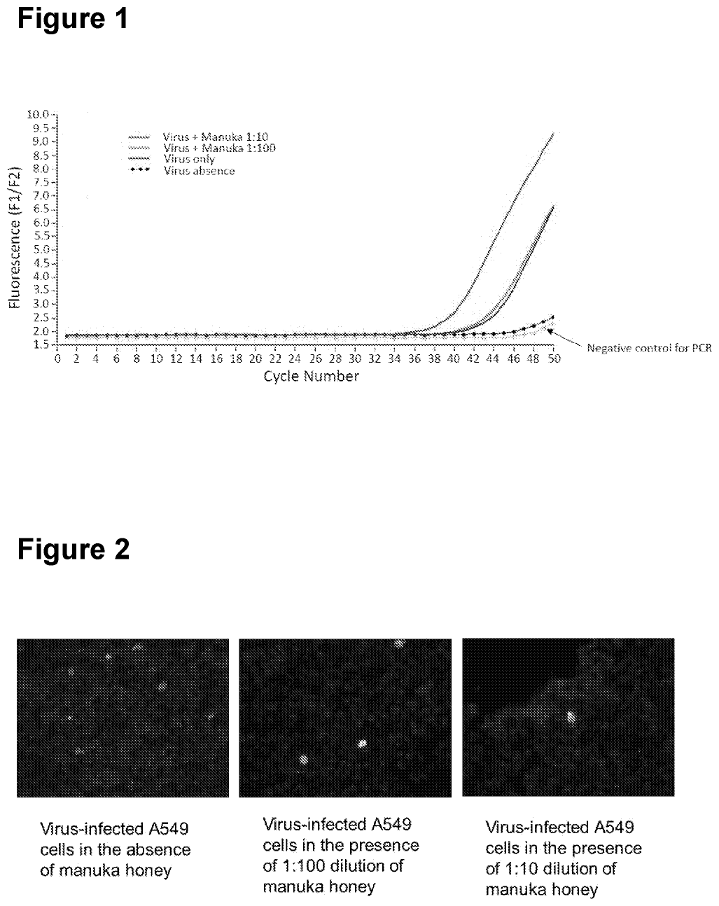

Samples of A549 cells were subjected to influenza A virus, H1N1, in vitro, both in the presence and absence of UMF16+ Manuka Honey comprising 550 mg/kg methylglyoxal. The honey was diluted to 1:10 and 1:100 in cell culture media and added to the cells. After incubating the cells for 7 days at 37±2° C. with 5% CO₂, the cells were harvested and the RNA was extracted and converted to cDNA. Quantitative polymerase chain reaction (qPCR) was used to measure the concentration of H1N1 RNA. The cells exposed to the honey had only about 10% of the viral count that the other cells had. This is illustrated in FIG. 1.

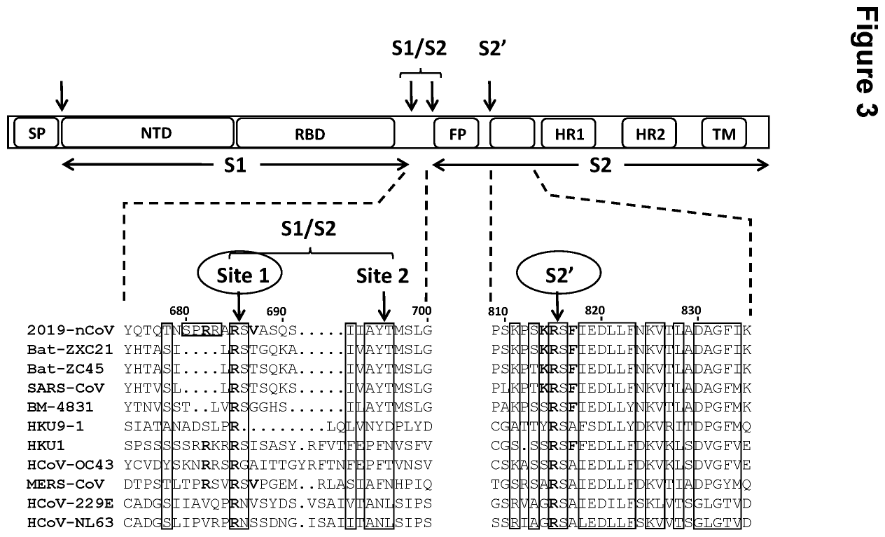

In a further test, A549 cells were treated with H1N1 virus in the presence and absence of UMF16+ Manuka Honey comprising 550 mg/kg methylglyoxal at 1:100 and 1:10 dilutions, prepared as above, and incubated for 7 days at 37±2° C. with 5% CO₂. Subsequently, an antibody raised against influenza A virus (H1N1) was applied to the samples, to identify the number of infected cells using immunohistochemistry, wherein the immunofluorescent signal identified in infected cells discriminated them from non-infected cells. The number of positive cells was counted under a microscope. The results are shown in FIG. 2, where the untreated cells rendered a virus indicating immunofluorescent signal of 7, compared to 3 and 1 for the 1:100 and 1:10 honey treated samples respectively. The uninfected cells are illustrated with red immunofluorescence in FIG. 2, due to uptake of Evans Blue dye used as a counterstain.¹ Obtained from Chemicon, Temecula, Calif., USA.

### SARS-CoV-1 & SARS-Co-V-2 Trials

Virucidal assays were carried out using UMF16+ Manuka Honey comprising 550 mg/kg methylglyoxal on SARS-CoV-1 and SARS-CoV-2 viruses. For this, Manuka Honey was diluted in sterile water to 1%, 3% and 10% w/v and the SARS-CoV viruses were added to these solutions and left for 1 hour along with sterile water as a negative control and 35% ethanol as a positive control. Following this contact step, a 10-fold dilution of the samples were made in cell culture media (minimal essential medium, supplemented with 2% w/v foetal bovine serum and 50% μg/mL gentamicin) and then 100 uL of each sample was applied to quadruplicate wells of plates containing confluent Vero 76 cells that were at a sub-confluent stage of growth (80-90% confluence). After incubating the cells for 6-7 days at 37±2° C. with 5% CO₂, the titre of the virus was determined with a CCID₅₀ end-point assay, calculated using the Reed-Muench (1948) equation. Three independent replicates of each sample were tested and the average and standard deviation were calculated. The cytopathic effect on cells was assessed under a microscope after an endpoint dilution in 96-well microplates.

The results for the treated samples were compared with the controls by one-way analysis of variance (ANOVA) with Dunnett's multiple comparison tests using GraphPad Prism (version 8) software.

The reduction in virus in the test wells was compared for the treated and control samples on a log reduction value (LRV) basis. Toxicity controls were tested with media not containing virus to determine whether the samples were toxic to cells. Neutralisation controls were tested to ensure that virus inactivation did not continue after the contact period and that residual sample in the titre assay plates did not inhibit growth and detection of surviving virus. This was done by adding toxicity samples to titre test plates and then spiking each well with a low amount of the virus that would produce an observable amount of cytopathic effect (CPE) during the incubation period. The trial results were as follows

TABLE 1
Virucidal activity of Manuka honey against SARS-CoV-1 after
a 1-hour contact time at 22 ± 2° C.

| Sample | Virus | |
|---|---|---|
| Concentration | (CCID₅₀/0.1 mL)^{a} | LRV^{b} |
| 10% manuka honey | 3.2 ± 0.4* | 1.1 |
| 3% manuka honey | 4.1 ± 0.2 | 0.2 |
| 1% manuka honey | 4.2 ± 0.6 | 0.1 |
| EtOH (35%) | 1.5 ± 0.2**** | 2.8 |
| Virus Control | 4.3 ± 0.3 | N/A |

^{a}Average of 3 replicates ± standard deviation
^{b}LRV is the log₁₀ reduction of virus compared to the virus control
** P < 0.01 and
****P < 0.0001 by one-way ANOVA and Dunnett's multiple comparison test, compared with untreated virus control (water)

TABLE 2
Virucidal activity of Manuka honey against SARS-CoV-2 after
a 1-hour contact time at 22 ± 2° C.

| Sample | Virus | |
|---|---|---|
| Concentration | (CCID₅₀/0.1 mL)^{a} | LRV^{b} |
| 10% manuka honey | 2.0 ± 0.5**** | 2.6 |
| 3% manuka honey | 2.6 ± 0.4*** | 2.0 |
| 1% manuka honey | 3.0 ± 0.5** | 1.6 |
| EtOH (35%) | <0.7**** | 3.9 |
| Virus Control | 4.6 ± 0.5 | N/A |

^{a}Average of 3 replicates ± standard deviation
^{b}LRV is the log₁₀ reduction of virus compared to the virus control
**P < 0.01,
***P < 0.001,
****P < 0.0001 by one-way ANOVA and Dunnett's multiple comparison test, compared with untreated virus control (water)

As illustrated in Tables 1 and 2, the ability of the two SARS viruses to infect the Vero 76 cells had been significantly inhibited in the honey containing samples.

### Antiviral Substances

The inventors consider that the replication of SARS- and MERS-CoVs viruses can be inhibited by compounds found in Manuka Honey that interfere with the action of the protease 3CLpro, and by compounds that interfere with the ORF 3a ion channel in SARS-CoV.

The inventors developed an assay using the 3CLpro-like protease expressed from a cDNA clone and the substrate Thr-Ser-Ala-Val-Leu-Gln-pNA (pNitroanilide), where the 3CLpro protease cleaves between the Gln-pNA bond to produce an increase in absorbance that can be measured at 390 nm. More specifically, the effect of Manuka Honey, methylglyoxal, phenolic acids and flavonoids (listed) were tested in 96-well plates on their ability to inhibit the activity of the 3CLpro protease to cleave the substrate, which was measured as a change in absorbance at 390 nm read on a plate reader It was found that the phenolic acids: 3-phenyllactic acid and p-coumaric acid and the flavonoids: quercetin, kaempferol, chrysin, luteolin pinocembrin, pinobanksin, hesperidin and galangin, each separately inhibit activity of 3CLpro protease and, therefore, replication of the viruses SARS-CoV and SARS-CoV-2.

The inventors consider that compounds found in Manuka Money, alone or in any combination, being methylglyoxyl 3-phenyllactic acid, p-coumaric acid, quercetin, kaempferol, chrysin, luteolin, pinocembrin, pinobanksin, hesperidin and galangin have antiviral affect against SARS- and MERS-CoVs, for example SARS-CoV and SARS-CoV-2.

### Methylglyoxal

As noted above, one aspect the invention comprises the use of methylglyoxal (MGO) in the preparation of an antiviral medicament for administering to a patient to treat, or prevent, infection by the virus (eg in a human). Optionally the virus comprises SARS-CoV-1, SARS-CoV-2 or MERS. The methylglyoxal may be carried and delivered in, or as a component of, honey (eg Manuka honey), but can also be carried or delivered in a suitably alternative way. For example the MGO may be carried in water with or without sugar, or in glycerol. The MGO may be delivered to fabric that forms part of an antiviral mask of the type described later in this specification.

In the context of MGO, the inventor(s) determined that three steps are required for a virus such as SARS-CoV-2 to gain entry into a host cell to replicate. Firstly, the receptor binding domain (RBD) on SARS-CoV-2 needs to recognise and engage the ACE2 receptors on the surface of a host cell. Secondly, site S1/S2 is required to be cleaved by the enzyme furin and, thirdly, site S2′ is required to be cleaved by the enzyme transmembrane protease, serine 2 (TMPRSS2) (FIG. 3). Binding to ACE2 receptors alone and/or cleavage of only one site compromises activation.

More specifically, FIG. 3 illustrates the sequence of SARS-CoV-2 protein with the key protease sites noted. Furin cleaves the spike protein at Site 1 and the transmembrane protease, serine 2 (TMPRSS2) cleaves the spike protein at Site S2′. Glycation of the arginine (R) residues proximal to the cleavage site blocks the action of those enzymes and prevents cleavage and, therefore, activation of the spike protein. Figure adapted from Bestle (2020)² and Coutard (2020)³. ²BESTLE, D., HEINDL, M. R., LIMBURG, H., VAN LAM VAN, T., PILGRAM, O., MOULTON, H., STEIN, D. A., HARDES, K., EICKMANN, M., DOLNIK, O., ROHDE, C., KLENK, H. D., GARTEN, W., STEINMETZER, T. & BOTTCHER-FRIEBERTSHAUSER, E. (2020). TMPRSS2 and furin are both essential for proteolytic activation of SARS-CoV-2 in human airway cells. Life Sci Alliance 3.³COUTARD, B., VALLE, C., DE LAMBALLERIE, X., CANARD, B., SEIDAH, N. G. & DECROLY, E. (2020). The spike glycoprotein of the new coronavirus 2019-nCoV contains a furin-like cleavage site absent in CoV of the same Glade. Antiviral Res 176, 104742.

To examine whether MGO is able to block the activity of furin at site S1 and TMPRSS2 at site S2′, two peptide substrates corresponding to sites S1 and S2′ were synthesised with a colorimetric (p-nitroaniline, pNA) C-terminal reporter (GL Biochem Ltd, Shanghai 200241, China). The two peptides required are provided below for Sites S1 and S2′:

Site S1 peptide (Peptide 1): Acetyl-SPRRAR↓pNA
Site S2′ peptide (Peptide 2): Acetyl-SKPSKR↓pNA

The principle of the assay was that the pNA substrates should be cleaved when furin or TMPRSS2 are added to the assay as this will result in a colour change. Trypsin was used as a surrogate for TMPRSS2 because both enzymes cleave the same motif (K/R↓) (Bestle et al., 2020). MGO is known to glycate numerous amino acids (arginine, lysine, histidine, cysteine, leucine, isoleucine, valine, aspartic acid, glutamic acid, alanine, proline, threonine, tyrosine, phenylalanine and serine) in the Maillard reaction, of which arginine is the most reactive (Takahashi, 1977). Glycation is the covalent attachment of a sugar, or in this case MGO, to a protein or lipid. It was determined that if MGO alone, or in Manuka Honey, glycates one or more of the residues and, in particular, the arginine (R) proximal to the cleavage site, then access to the enzyme may be blocked, which would prevent cleavage of pNA and result in no colour change.⁴

Active recombinant human furin and trypsin were purchased from BioLegend (San Diego, USA) and (Sigma Aldrich, USA), respectively. MGO was purchased from Sigma-Aldrich (Auckland, New Zealand). An assay was carried out in 96-well plates with 400 μM of peptide substrate, 8 mM MGO and 5 nM each of furin and trypsin with incubation times of 0-240 min. The colour change is read on a plate reader at 410 nm. ⁴TAKAHASHI, K. (1977). The reactions of phenylglyoxal and related reagents with amino acids. J Biochem 81, 395-402.

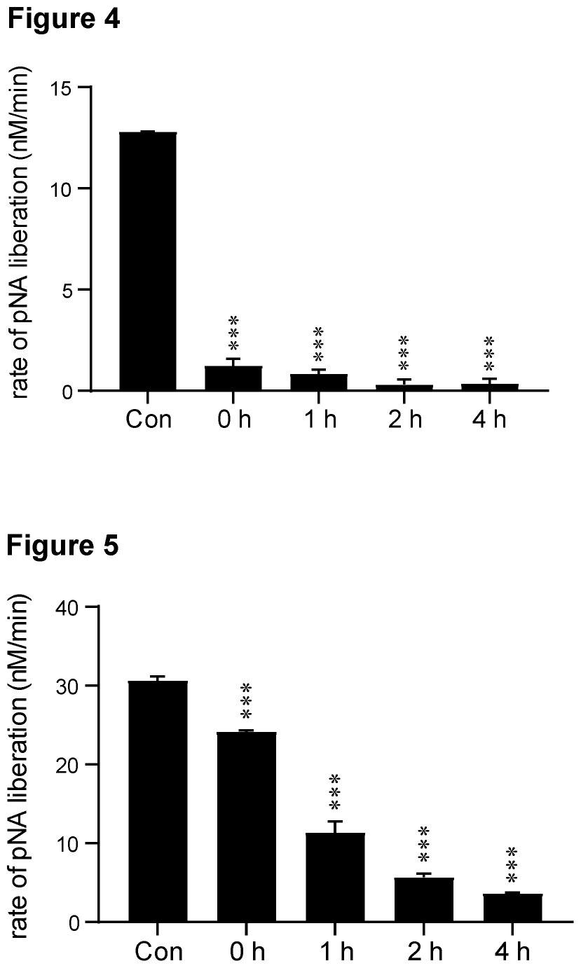

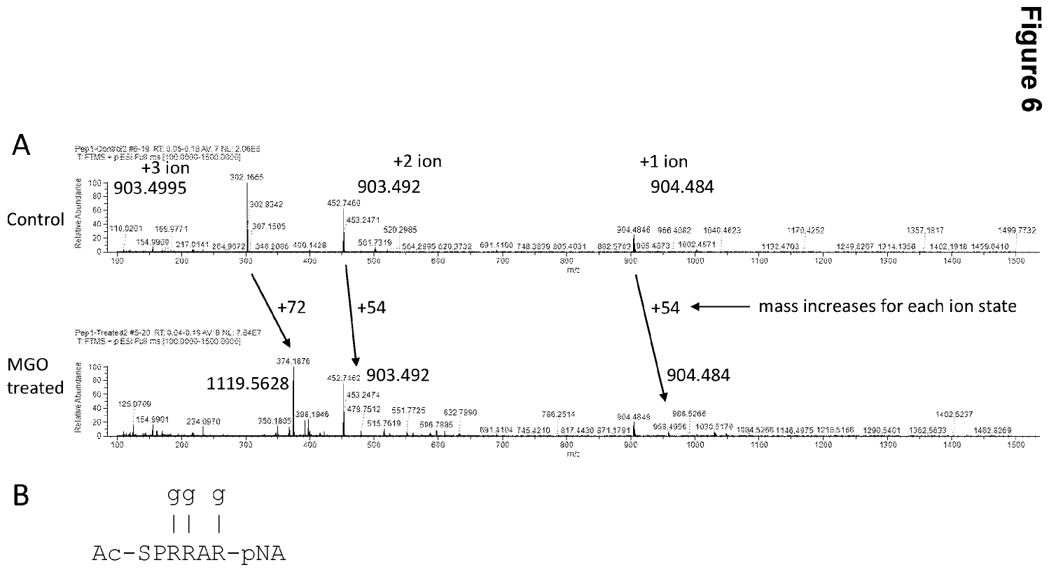

It was found that MGO prevents cleavage and activation of peptide 1 by furin (FIG. 4) and of peptide 2 by trypsin (FIG. 5). The inhibition was rapid and resulted in a 90% reduction in activation of peptide 1 and a 21% reduction of peptide 2 at time 0. The extent of the inhibition progressed to further inhibit action of both peptides by 97% for peptide 1 and 88% for peptide 2 at 4 h.

FIG. 4 illustrates the rate of liberation of pNA after addition of the enzyme furin (5 nM) to peptide 1 (400 μM) that had been incubating with MGO (8 mM) for the times stated. There is a 90% reduction in the amount of pNA liberated at time 0 indicating that the action of MGO to glycate the peptide and prevent access to the enzyme is instant. The inhibition increases to 97% at 3 and 4 h. Asterisks indicate significance from untreated controls (***P<0.001). Further, FIG. 5 illustrates the rate of liberation of pNA after addition of the enzyme trypsin (5 nM) to peptide 2 (400 μM) that had been incubating with MGO (8 mM) for the times stated. There is a 21% reduction in the amount of pNA liberated at time 0 indicating that the action of MGO to glycate the peptide and prevent access to the enzyme is instant and progresses rapidly. The inhibition increases to 88% at 4 h. Asterisks indicate significance from untreated controls (***P<0.001).

### Confirmation of Glycation by Mass Spectrometry

The Mass Spectra of peptides 1 and 2 were examined before and after addition of MGO (8 mM MGO added to 400 μM of peptide for 1 h). Peptide samples were prepared for analysis by diluting 50 μL of the reaction in 950 μL of 10% acetonitrile/0.2% formic acid. Samples were analysed using a Q Exactive™ Q Exactive Orbitrap™ mass spectrometer (UHPLC-Orbitrap-MS/MS, Thermo Fisher Scientific, San Jose, USA) using electrospray ionisation (ESI). Samples were injected directly into the instrument and full scan spectra (full scan data-dependent acquisition) were obtained under ESI positive and ESI negative mode. A 50/50 mobile phase was used, consisting of acetonitrile and 0.2% formic acid and the flow rate was 0.5 mL/min for a runtime of 1 min throughout the separation. The full scan resolving power was 70,000 MWHM at m/z 200. A scan range of m/z 100-1500 was chosen with the automatic gain control (AGC) set to 1e6, with an injection time of 100 ms.

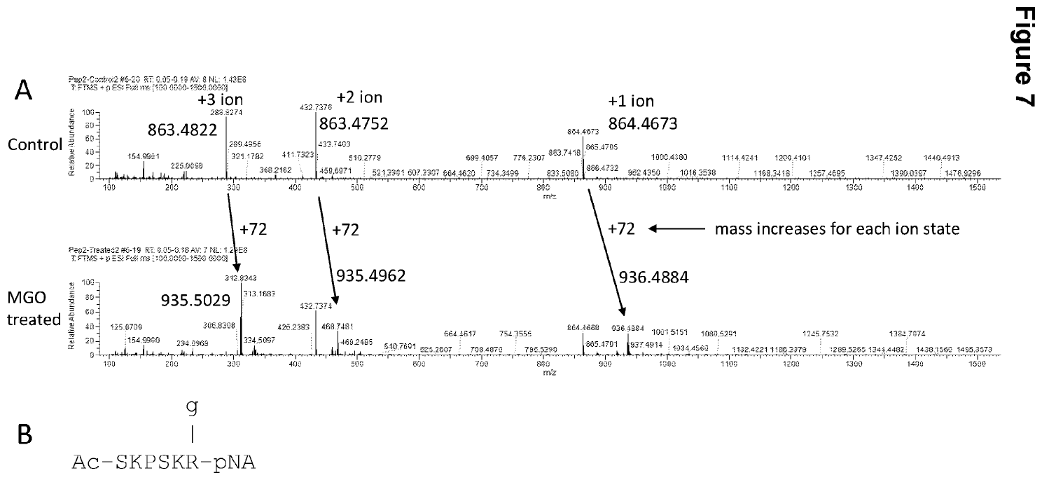

The mass spectrometry data show that only the arginine residues were glycated by MGO, which is consistent with the greater reaction of those residues to MGO, despite the presence of other possible reactions (lysine, serine and alanine) (FIGS. 6 and 7). The 54 and 72 Da increases in the size of the mass spectra are consistent with the transient compound dihydroxyimidazolidine (72 Da) formed by the addition of MGO to arginine and the more stable glycated compounds MG-H1-3 (54 Da) after removal of water (Rabbani et al., 2016⁵).

FIG. 6 illustrates at. (A) mass spectra showing the difference in masses after treating peptide 1 (S1) with MGO. The masses increase by 72 or 54 Da after addition of MGO, which accord with the transient compound dihydroxyimidazolidine after the glycation of arginine (72 Da) and the more stable glycated products (MG-H1-3) after removal of water (54 Da). And at (B) is shown increased mass is consistent with glycation (g) of the three arginine residues.

FIG. 7 illustrates at (A) the mass spectra showing the difference in masses after treating peptide 2 (S2′) with MGO. The masses increase by 72 Da after addition of MGO, which accords with the transient compound dihydroxyimidazolidine after the glycation of arginine. Item (B) illustrates that increased mass is consistent with glycation (g) of the arginine residue.

Based on the above, the inventors have discovered that MGO glycates arginine residues in the S1 and S2′ regions of the SARS-CoV-2 spike protein, thereby blocking access to, and cleavage of those sites by furin and trypsin and/or TMPRSS2. The data indicate that medical grade Manuka Honey has a virucidal action on SARS-CoV-1 and SARS-CoV-2.

### Antiviral Masks

As indicated above, the inventors have determined that using a mask covering the nose and mouth, where the air breathed in has to pass through a barrier in the form of a filter, provides enhanced antiviral protection if the air engages a suitable amount of Manuka Honey as it passes through the filter. Preferably the filter has a pore size and path sufficient to allow the passage of air, but not to allow the passage of virus, at least when the filter is loaded with honey. ⁵RABBANI, N., ASHOUR, A. & THORNALLEY, P. J. (2016). Mass spectrometric determination of early and advanced glycation in biology. Glycoconj J 33, 553-568.

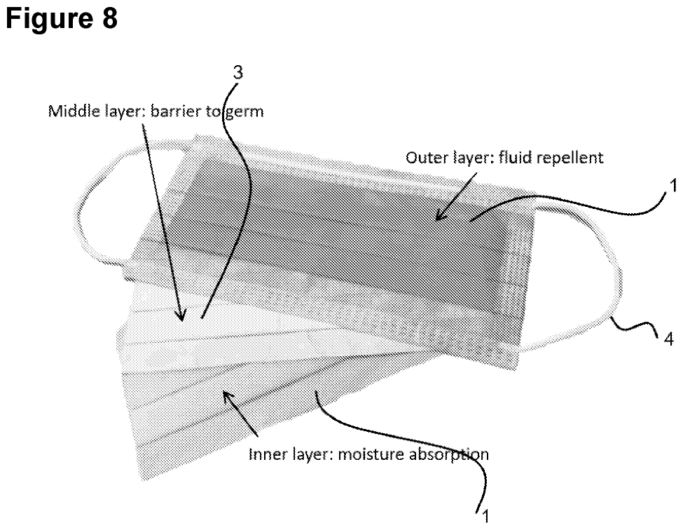

In a preferred embodiment the mask is formed as indicated in FIG. 8, having an outer sheet 1 of fluid repellent material, an inner moisture absorbent sheet 2 and an absorbent fabric barrier sheet 3 between the two that is impregnated with Manuka Honey. The mask is worn so that the sheets cover a person's nose and mouth. The user is able to breath air in through the sheets, and also to exhale through them. The mask is held in place by way of elastic straps 4 that engage the user's ears.

Preferably the Manuka Honey sits in interstitial spaces of the fabric barrier sheet 2. Over time the fibers making up the barrier 3 swell with moisture to provide a greater surface area for the honey coating, and a more difficult path for a virus to pass through. The honey provides an antiviral affect against virus that comes into contact with it.

A Manuka Honey impregnated barrier 3 can also be used in a similar way with masks having more, or fewer, layers than described for FIG. 4 (eg one or two layers only). In some embodiments the mask may be in the form of a respirator and the barrier 3 may be positioned across an air channel of the respirator to provide antiviral affect in substantially the same manner described above.

Preferably masks according to the invention are such that the barrier comprises Manuka Honey loaded polypropylene, eg meltblown polypropylene and/or superabsorbent fibre (SAF). An example of SAF is a polyacrylate fiber with cross linked sections of carboxyl groups. A further example is as described and/or claimed in US patent specification No. 2016/0158403 (Manukamed).

While some preferred embodiments of the invention have been described by way of example it should be appreciated that modifications and improvements can occur without departing from the scope of the following claims.

In terms of disclosure, this document hereby discloses each item, feature or step mentioned herein in combination with one or more of any of the other item, feature or step disclosed herein, in each case regardless of whether such combination is claimed.

1.-51. (canceled)

52. A method for treating a viral disease caused by one or more of SARS-CoV-1, SARS-CoV-2 or MERS virus, comprising applying Manuka Honey, directly or via a medicament, to the periphery of tissue on which the virus is present.

53. A method according to claim 52, wherein the disease is COVID-19.

54. A method according to claim 52, wherein the medicament is applied onto the tissue topically.

55. A method according to claim 52, wherein the medicament has the Manuka Honey in diluted form and is applied onto the back of the throat, nasal passages or mouth of a patient suffering from the viral disease.

56. A method according to claim 52, wherein the medicament comprises the Manuka honey in solution.

57. A method according to claim 52, wherein the medicament is delivered to the tissue as a spray or aerosol.

58. A method according to claim 52, wherein the Manuka Honey comprises methylglyoxal (MGO) at approximately 100-1,000 mg/kg of the honey.

59. A method according to claim 52, wherein the Manuka Honey comprises methylglyoxal (MGO) at ≥550 mg/kg of the honey.

60. A respiratory mask comprising a barrier permeable to air, the barrier having sufficient Manuka Honey for substantially disinfecting a virus in air when breathed in or otherwise passed through the barrier while the mask is worn, wherein the virus is SARS-CoV-1, SARS-CoV-2 or MERS.

61. A respiratory mask according to claim 60, wherein the Manuka Honey is effective in disinfecting viruses that cause COVID-19 disease.

62. A respiratory mask according to claim 60, wherein the Manuka Honey comprises methylglyoxal (MGO) at 100-1,000 mg/kg of the honey.

63. A respiratory mask according to claim 60, wherein the Manuka Honey comprises methylglyoxal (MGO) at ≥550 mg/kg of the honey.

64. A respiratory mask according to claim 60, wherein the Manuka Honey comprises methylglyoxal (MGO) at ≥700 mg/kg of the honey.

65. A respiratory mask according to claim 60, wherein the barrier comprises fibrous sheet or pad that holds a layer of Manuka honey.

66. A method of treating or preventing COVID-19 disease in a patient by administering to the periphery of tissue of the patient an antiviral medication comprising one or more of the substances:
a) methylglyoxal;
b) 3-phenyllactic acid;
c) p-coumaric acid;
d) pinocembrin;
e) pinobanksin; and
f) chrysin;
in a quantity sufficient to treat or prevent infection of the patient by one or more of SARS-CoV-1, SARS-CoV-2 and MERS.

67. A method according to claim 66, wherein the virus causes COVID-19 disease.

68. A method according to claim 66, wherein the medicament comprises honey in diluted form and is applied topically onto the tissue.

69. A method according to claim 66, wherein the honey is Manuka Honey.

70. A method according to claim 69, wherein the medicament has the Manuka Honey in diluted form and is applied onto the back of the patient's throat, nasal passages or mouth.

71. A method according to claim 66, wherein the medicament comprises the Manuka Honey in solution.

72. A method according to claim 66, wherein the medicament is delivered to the tissue as a spray.

73. A method according to claim 66, wherein the medicament comprises Manuka Honey having methylglyoxal (MGO) at 100-1,000 mg/kg of the honey.
